(19) United States
(12) Patent Application Publication
(10) Pub. No.: US 2023/0263669 A1
(43) Pub. Date: Aug. 24, 2023

(54) EXTRUSION OF ELASTIC ONTO A PRODUCT

(71) Applicant: NORDSON CORPORATION, WESTLAKE, OH (US)

(72) Inventors: Frank Jones, Lakewood, OH (US); Andreas Bornkessel, Adendorf (DE)

(21) Appl. No.: 18/004,076

(22) PCT Filed: Jul. 7, 2021
(86) PCT No.: PCT/US2021/040595

§ 371 Date: Jan. 3, 2023

Related U.S. Application Data
(60) Provisional application No. 63/049,205, filed on Jul. 8, 2020.

(51) Int. Cl. A61F 13/15; A61F 13/49

(52) U.S. Cl. CPC A61F 13/15747; A61F 13/15593; A61F 13/4902; A61F 2013/15861; A61F 2013/49022

(57) ABSTRACT

In one example, a method of fabricating a product, such as a diaper, includes a step of folding a substrate of the product to form a plurality of pleats in the substrate. The method includes a step of applying a liquified elastic to the substrate such that the liquified elastic adheres to the substrate and extends across the plurality of pleats. The method includes a step of allowing the liquified elastic to solidify on the substrate.

### CROSS REFERENCE TO RELATED APPLICATIONS

This application is a National Stage Application of International Patent App. No. PCT/US2021/040595, filed Jul. 7, 2021, which claims the benefit of U.S. Provisional Application No. 63/049,205, filed Jul. 8, 2020, the entire disclosures of both of which are hereby incorporated by reference as if set forth in their entirety herein.

### TECHNICAL FIELD

The present disclosure relates generally to extrusion of elastic and, in particular, to extrusion of elastic onto a product such as a hygiene product or garment.

### BACKGROUND

Conventionally, elastic is formed separately from a product such as a hygiene product or garment, and the elastic is subsequently attached to the product by adhering the elastic to the product, sewing the elastic to the product, or trapping the elastic in a seam. Sometimes, the elastic is formed on a backing sheet and is subsequently removed from the backing sheet for assembly into the product. Other times, the elastic is formed without a backing sheet such that the elastic is free from attachment to any substrate, the elastic is packaged, and then the elastic is subsequently removed from the packaging for assembly into the product. Commonly, the elastic is wound on a roll when it is formed and must be subsequently unwound from the roll for assembly into the product.

### BRIEF DESCRIPTION OF THE DRAWINGS

The following description of the illustrative examples may be better understood when read in conjunction with the appended drawings. It is understood that potential examples of the disclosed systems and methods are not limited to those depicted.

FIG. 1 shows a simplified schematic diagram of a system according to one example that comprises an elastic dispensing system and a substrate handling system;

FIG. 2 shows perspective, front, and side views of a manifold of an applicator of the elastic dispensing system of FIG. 1 according to one example;

FIG. 3 shows an image of an applicator applying elastic strands to a substrate according to one example;

FIG. 4 shows a top view of a substrate according to one example having a plurality of pleats and a plurality of elastic strands adhered to the substrate across the pleats;

FIG. 5 shows an end view of the substrate of FIG. 4;

FIG. 6 shows an end view of first and second substrates adhered to one another by an elastic strand; and

FIG. 7 shows a perspective view of a diaper according to one example having a plurality of pleats and a plurality of elastic strands adhered to the substrate across the pleats.

### DETAILED DESCRIPTION

Conventional methods of attaching elastic to a product can result in excess waste in the form of the backing material, packaging, and/or rolls that are employed to contain the elastic until the elastic is assembled into the product. Further, conventional methods of attaching elastic to a product require additional machinery or manpower to remove the backing material and/or packaging, and/or to unwind the elastic from the rolls. Disclosed herein are methods and systems that can apply elastic to products without creating such excess waste and without requiring additional machinery or manpower to unwind elastic from rolls. In general, the methods and systems apply liquified elastic directly to a product through an adhesive applicator as the product, or a portion thereof, is being manufactured. Thus, elastic strands (also referred to as filaments) are formed directly on a substrate of the product. This is in contrast to conventional methods, wherein elastic strands are formed separately from the product and then subsequently attached to the product.

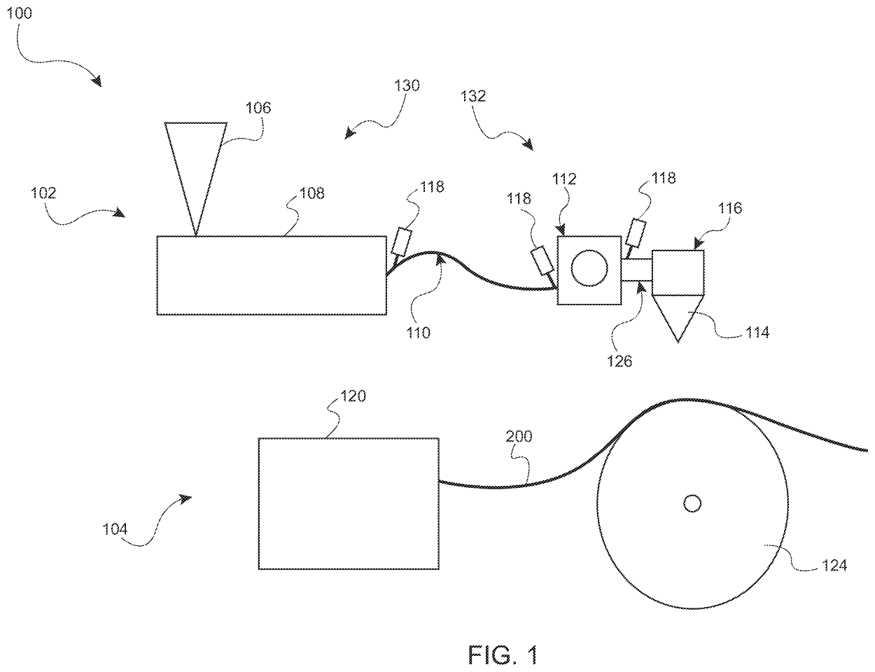

Turning to FIGS. 1 to 3, a system 100 according to one example comprises an elastic dispensing system 102 and a substrate handling system 104. The elastic dispensing system 102 can comprise a melting system 130 and an applicator 132. The melting system 130 can comprise a melter 108 that is configured to liquefy a solid elastomer. In one example, the melter 108 can be an extruder that is configured to extrude solid elastic and output liquified elastic. The solid elastomer can be in the form of pellets, blocks, or another suitable solid form. The melting system 130 can comprise a hopper 106 that is configured to feed solid elastomer to the melter 108. The elastomer can be any suitable elastomer, such as a thermoplastic elastomer.

The applicator 132 comprises at least one nozzle outlet 114 that is configured to dispense a strand of liquified elastic onto a substrate 200 of a product. The substrate 200 can be a woven or non-woven substrate. The at last one nozzle outlet 114 is supported over the substrate 200. The at least one nozzle outlet 114 can be spaced from the substrate 200, and therefore, the at least one nozzle outlet 114 can be considered a non-contact nozzle outlet since it does not contact the substrate. The applicator 132 comprises at least one dosing device 112 that is configured to receive liquified elastic from the melter 108 and provide liquified elastic to the at least one nozzle outlet 114. The at least one dosing device 112 can be a valve, such as (without limitation) a solenoid valve, a pneumatic valve, or a piezoelectric valve, a pump, or any suitable metering device. The elastic dispensing system 102 can comprise at least one conduit 110 that communicates the liquified elastic from the melting system 130 to the applicator 132. The at least one conduit 110 can be, for example, a flexible hose. The elastic dispensing system 102 can comprise at least one, such as a plurality, of pressure sensors 118 that are configured to monitor a pressure of the liquified elastic as the liquified elastic is distributed through the elastic dispensing system 102.

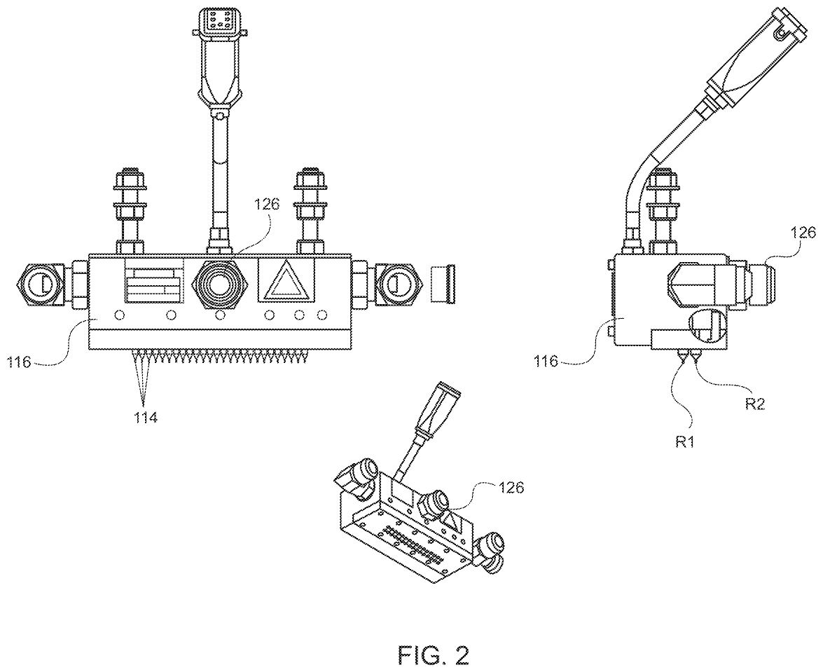

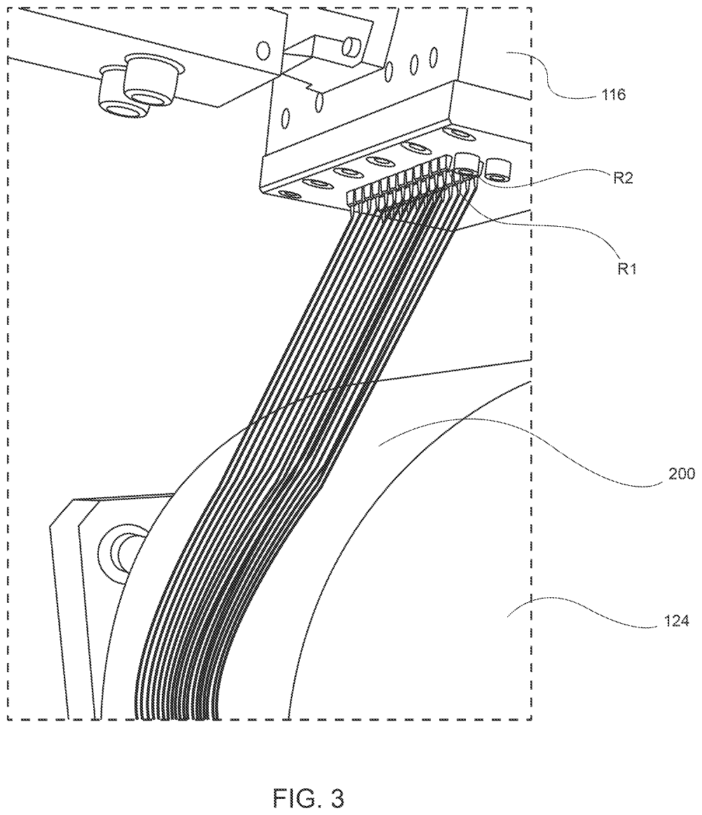

With reference to FIGS. 2 and 3, in some examples, the applicator 132 can comprise a plurality of nozzle outlets 114, each configured to dispense a separate strand of liquified elastic onto the substrate 200. For example, the applicator 132 can comprise two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, or more nozzle outlets 114. The applicator 132 can comprise a manifold 116 having an inlet 126 that receives the liquified elastic, and at least one distribution channel therein (not shown) that distributes the liquified elastic to the nozzle outlets 114. In some examples, the manifold 116 can be heated so as to heat the liquified elastic.

The nozzle outlets 114 can be arranged in at least one row of nozzle outlets 114, where the nozzle outlets 114 in each row are spaced from one another along a row direction. In some examples, the nozzle outlets 114 can be arranged in two or more rows of nozzle outlets 114, where the rows are spaced from one another along a column direction that is perpendicular to the row direction. The nozzle outlets 114 in each row can define spaces therebetween. The nozzle outlets 114 in one row Ri can be aligned along the column direction with the spaces between the nozzle outlets 114 in an adjacent row Rz. In other words, the nozzle outlets 114 in the rows can be staggered from one another so that the nozzle outlets 114 are not aligned with one another along the column direction.

As shown in FIGS. 2 and 3, in some examples, the applicator 132 can include at least one nozzle, where each nozzle defines a nozzle outlet 114. In alternative examples (not shown), the applicator 132 can include a slot nozzle that defines an elongate slot, where the slot nozzle has at least one shim that divides the elongate slot into a plurality of nozzle outlets 114.

Returning to FIG. 1, the substrate handling system 104 can comprise a substrate support device 124 having a surface that supports the substrate 200 below the at least one nozzle outlet 114. In one example, the substrate support device 124 can comprise a roller. The substrate handling system 104 can optionally include a substrate folding system 120. The substrate folding system 120 can be a machine that is configured to fold the substrate to form a plurality of pleats 210 therein. Alternatively, the substrate can be folded by an operator. In some examples, the substrate handling system 104 can be a hygiene product forming machine that is configured to form a hygiene product such as a diaper. In some examples, the diaper can be an adult diaper. In some examples, the substrate handling system 104 can be a diaper forming machine manufactured by Fameccanica or other manufacturer.

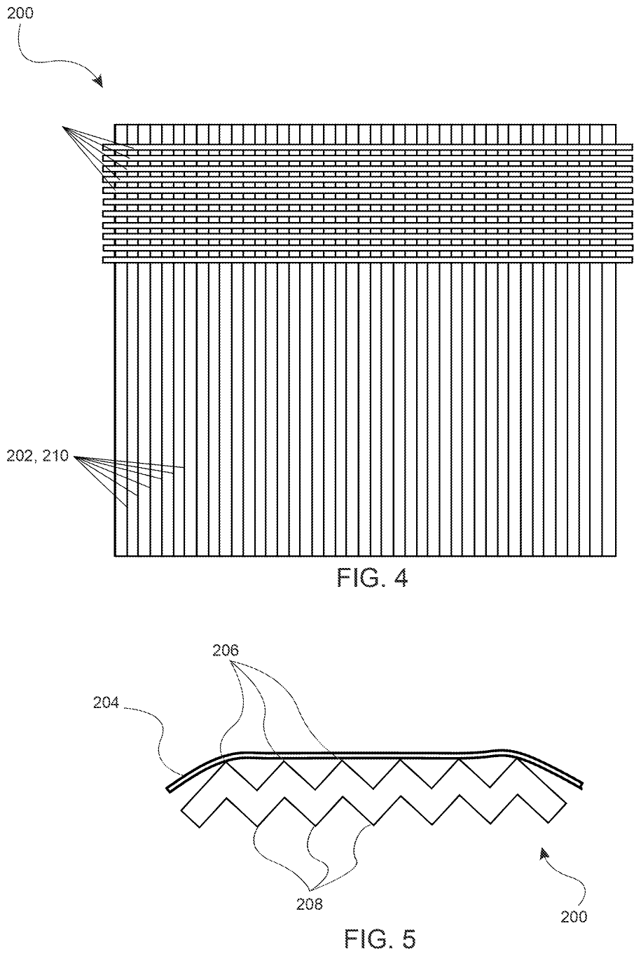

Turning now to FIGS. 4 and 5, a substrate 200 of a product has a plurality of pleats 210 therein that are defined by a plurality of fold lines 202. In FIGS. 4 and 5, the substrate 200 is folded to define accordion pleats 210. However, it will be understood that, in alternative examples, the substrate 200 can be folded into other style pleats 210 such as (without limitation) knife pleats, sunray pleats, box pleats, and inverted box pleats. The fold lines 202 can be parallel to one another as shown or can be angularly offset from one another (e.g., non-parallel). The fold lines 202 can define a plurality of peaks 206 and a plurality of valleys 208. Individual ones of the valleys 208 can be spaced between adjacent peaks 206. Likewise, individual ones of the peaks 206 can be spaced between adjacent valleys 208.

The substrate 200 has at least one elastic strand 204 adhered thereto that extends across the pleats 210. Each elastic strand 204 is angularly offset from the fold lines 202. In some examples, each elastic strand 204 can be substantially perpendicular to the fold lines 202, although, in other examples, each elastic strand 204 can extend at an angle other than 90 degrees relative to the fold lines 202. The elastic strands 204 can be spaced from one another. In some examples, the elastic strands 204 can be substantially parallel to one another, while in other examples, the elastic strands 204 can be angularly offset (e.g., non-parallel) to one another. Each elastic strand 204 can be adhered to the peaks 206 of the substrate 200 such that the elastic strand 204 extends over and is spaced from the valleys 208. Each elastic strand 204 is adhered to the substrate 200 by the elastic material that forms the elastic strands, and not by a separate adhesive that is adhered to the elastic strand 204.

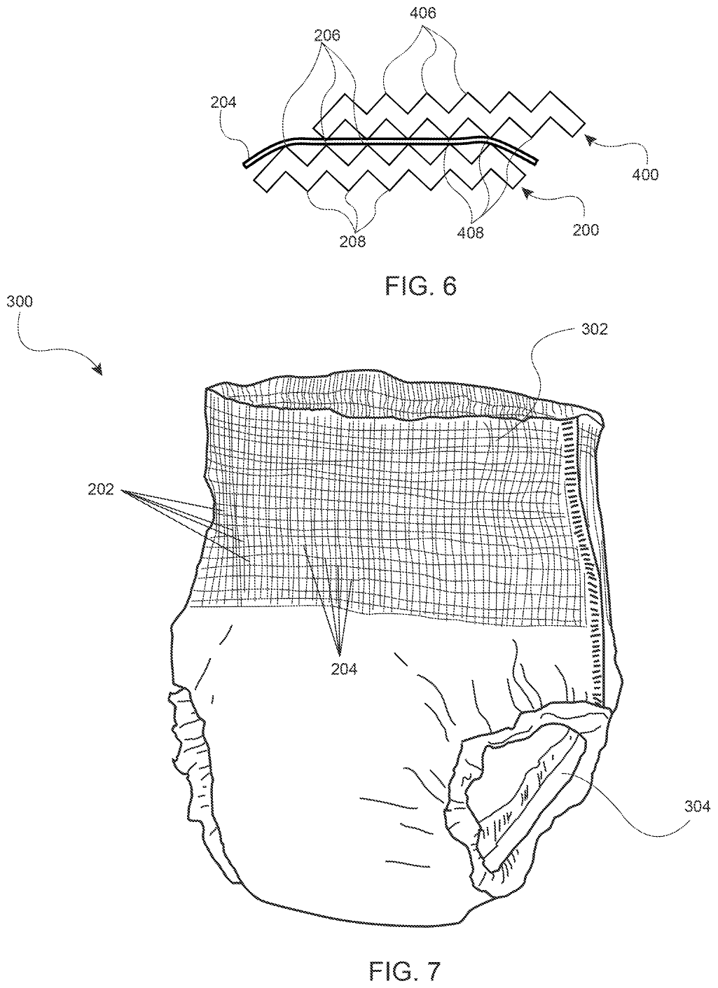

Turning to FIG. 6, in some examples, the at least one elastic strand 204 can be used to adhere a second substrate 400 to the first substrate 200. The first substrate 200 can be considered to be a carrier layer, and the second substrate 400 can be considered to be a cover layer. In some examples, the cover layer can be positioned so as to be closer to the skin. The second substrate 400 can have a plurality of pleats therein that are defined by a plurality of fold lines. The second substrate 400 can be folded to define accordion pleats or can be folded into other style pleats such as (without limitation) knife pleats, sunray pleats, box pleats, and inverted box pleats. The fold lines can be parallel to one another as shown or can be angularly offset from one another (e.g., non-parallel). The fold lines can define a plurality of peaks 406 and a plurality of valleys 408. Individual ones of the valleys 408 can be spaced between adjacent peaks 406. Likewise, individual ones of the peaks 406 can be spaced between adjacent valleys 408.

The at least one elastic strand 204 can be applied between the first substrate 200 and the second substrate 400. In some examples, individual ones of the peaks 206 of the first substrate 200 can be adhered to individual ones of the valleys 408 of the second substrate 400. Thus, the elastic strand 204 can define a plurality of attachment points, each attachment point adhering one peaks 206 of the first substrate 200 to one valley 408 of the second substrate 400.

Referring to FIG. 7, in one example, the product can be a diaper 300, such as an adult diaper, and the substrate can be a layer of the diaper 300. In the orientation of FIG. 7, the fold lines 202 extend in the vertical direction, and the elastic strands 204 extend along the horizontal direction. In some examples, the elastic strands 204 can be adhered to a waistband 302 of the diaper 300. In some examples, the elastic strands 204 can be adhered to a portion of the diaper 300 that is above leg holes 304 of the diaper 300. In some examples, the elastic strands 204 can be adhered to a portion of the diaper 300 that is between the waistband 302 and the leg holes 304. In some examples, the elastic strands 204 can be applied around the leg holes 304.

Referring generally to FIGS. 1-6, a method of fabricating a product comprises a step of folding a substrate 200 of the product to form a plurality of pleats 210 in the substrate 200. The method further comprises applying a liquified elastic to the substrate 200 such that the liquified elastic adheres to the substrate 200 and extends across the plurality of pleats 210. The method yet further comprises allowing the liquified elastic to solidify on the substrate 200.

In some examples, the applying step can comprise applying an elastic strand 204 of the liquified elastic to the substrate 200, the elastic strand 204 of liquified elastic extending across the plurality of pleats 210. In some examples, the applying step can comprise applying a plurality of elastic strands 204 of the liquified elastic to the substrate 200, wherein each elastic strand 204 of liquified elastic extends across the plurality of pleats 210. The elastic strands 204 of liquified elastic can be applied so as to be spaced from one another. For example, the strands of liquified elastic 204 can be applied to be substantially parallel to one another.

In some examples, the applying step comprises applying the liquified elastic across the peaks 206 of the pleats 210 such that the liquified elastic extends over and is spaced from the valleys 208 of the pleats. Preferably, the liquified elastic is hot enough that it is tacky and can stick to the substrate 200. Preferably, the liquified elastic is cool enough that it can stretch between peaks 206 in the substrate 200 without falling into the valleys 208.

The folding step can comprise folding the substrate 200 to form at least one accordion pleat or other type of pleat in the substrate 200. The substrate 200 can be a substrate of a hygiene product, such as a diaper 300, such as a child or adult diaper. In some examples, the applying step can comprise applying the liquified elastic to a waistband 302 of the diaper 300. In some examples, the applying step can comprise applying the liquified elastic to a portion of the diaper that is below a waistband 302 of the diaper 300 and above leg holes 304 of the diaper 300. In some examples, the applying step can comprise applying the liquified elastic around the leg holes 304 of the diaper 300.

Although some of the figures show the application of a thin bead of liquified elastic to a substrate, examples of the disclosure are not limited to thin beads. In alternative examples, the applicator can be configured to dispense wider strips of liquified elastic or patterns (e.g., swirls) of elastic other than a straight line. For example, the applicator can comprise a slot nozzle that is elongate so as to dispense a wider strip of liquified elastic.

It should be noted that the illustrations and descriptions of the examples shown in the figures are for exemplary purposes only, and should not be construed limiting the disclosure. One skilled in the art will appreciate that the present disclosure contemplates various examples. Additionally, it should be understood that the concepts described above with the above-described examples may be employed alone or in combination with any of the other examples described above. It should further be appreciated that the various alternative examples described above with respect to one illustrated example can apply to all examples as described herein, unless otherwise indicated.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more examples or that one or more examples necessarily include these features, elements and/or steps. The terms “comprising,” “including,” “having,” and the like are synonymous and are used inclusively, in an open-ended fashion, and do not exclude additional elements, features, acts, operations, and so forth.

While certain examples have been described, these examples have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

It will be understood that reference herein to “a” or “one” to describe a feature such as a component or step does not foreclose additional features or multiples of the feature. For instance, reference to a device having or defining “one” of a feature does not preclude the device from having or defining more than one of the feature, as long as the device has or defines at least one of the feature. Similarly, reference herein to “one of” a plurality of features does not foreclose the invention from including two or more, up to all, of the features. For instance, reference to a device having or defining “one of a X and Y” does not foreclose the device from having both the X and Y.

1. A method of fabricating a product, the method comprising:
folding a substrate of the product to form a plurality of pleats in the substrate;
applying a liquified elastic to the substrate such that the liquified elastic adheres to the substrate and extends across the plurality of pleats; and
allowing the liquified elastic to solidify on the substrate.

2. The method of claim 1, wherein the applying comprises applying a strand of the liquified elastic to the substrate, the strand of liquified elastic extending across the plurality of pleats.

3. The method of claim 1, wherein the applying comprises applying a plurality of strands of the liquified elastic to the substrate, wherein each strand of liquified elastic extends across the plurality of pleats.

4. The method of claim 3, wherein the strands of liquified elastic are spaced from one another.

5. The method of claim 3, wherein the strands of liquified elastic are substantially parallel to one another.

6. The method of claim 1, wherein the step of folding the substrate comprises forming at least one accordion fold in the substrate.

7. The method of claim 1, wherein the substrate is a substrate of a hygiene product.

8. The method of claim 7, wherein the hygiene product is a diaper.

9. The method of claim 8, wherein the hygiene product is an adult diaper.

10. The method of claim 8, wherein the applying comprises applying the liquified elastic to a portion of the diaper that is below a waistband of the diaper.

11. The method of claim 10, wherein the applying comprises applying the liquified elastic to a portion of the diaper that is above leg holes of the diaper.

12. The method of claim 8, wherein the applying comprises applying the liquified elastic to a waistband of the diaper.

13. The method of claim 8, wherein the applying comprises applying the liquified elastic to around leg holes of the diaper.

14. The method of claim 1, wherein the pleats define a plurality of peaks and a plurality of valleys, and the applying comprises applying the liquified elastic across the peaks such that the liquified elastic extends over and is spaced from the valleys.

15. A system configured to form at least a portion of a product, the system comprising:
a machine configured to fold a substrate of the product to form a plurality of pleats in the substrate; and
an applicator configured to apply a liquified elastic to the substrate such that the liquified elastic adheres to the substrate and extends across the plurality of pleats.

16. The system of claim 15, wherein the machine is a diaper forming machine.

17. The system of claim 15, wherein the applicator comprises a plurality of nozzle outlets, each configured to dispense a separate strand of liquified elastic onto the substrate.

18. The system of claim 17, wherein the plurality of nozzle outlets comprises at least one row of nozzle outlets.

19. The system of claim 15, comprising a melting system configured to liquefy elastic and provide the liquified elastic to the applicator.

20. The system of claim 15, wherein the applicator is supported over the substrate.

21. A product, comprising:
a substrate folded so as to define a plurality of pleats therein; and
at least one elastic strand that extends across the plurality of pleats, wherein the at least one elastic strand is adhered to the substrate by an elastic material that forms the at least one elastic strand, and not by a separate adhesive adhered to the elastic strand.
